(19) United States
(12) Patent Application Publication
(10) Pub. No.: US 2002/0186850 A1
(43) Pub. Date: Dec. 12, 2002

(54) Rotating control barb for use with an electronic stethoscope

(76) Inventors: Richard J. Deslauriers, Woodbury, CT (US); Robert T. Potash, S. Windsor, CT (US)

Correspondence Address: RICHARD J. DESLAURIERS, 143 WOLCOTT ROAD, WOLCOTT, CT, 06716, US

(21) Appl. No.: 09/877,172

(22) Filed: Jun. 11, 2001

(51) Int. Cl. A61B 7/04; A61B 7/02

(52) U.S. Cl. 381/67; 181/131; 600/528

(57) ABSTRACT

This invention relates to the field of stethoscopes and specifically a method for controlling the functions and operations of a digital stethoscope. The disclosed invention portrays a rotating control barb extending from the head of a digital stethoscope. The disclosed invention allows a user to selects between multiple features that are available on a digital stethoscope by rotating the control barb 360° around the circumference of the control barb to multiple stopping points, whereby said rotation along the 360° circumference sets the electronic features within a digital stethoscope. A method of controlling the functions of a digital stethoscope without adding cumbersome or difficult to use controls to the stethoscope head, and whereby external connections are not required. Furthermore, the disclosed method does not hamper the quality or affect the operations of the stethoscope head.

### CROSS-REFERENCE TO RELATED APPLICATIONS

### STATEMENT REGARDING FEDERALLY SPONSORED RESEARCH OR DEVELOPMENT

Not Applicable

### REFERENCE TO A MICROFICHE APPENDIX

Not Applicable

### BACKGROUND OF THE INVENTION

1. Field of the Invention

This invention relates to the field of stethoscopes, and in particular a method of controlling various operations of a digital stethoscope.

2. Description of the Related Art

The use of stethoscopes has long been a necessary part to the physical examinations of patients by their doctors. The field of stethoscope manufacturing includes those conventional stethoscopes that operate solely on the principles of sound travel and the coupling of sound waves through an acoustic cavity. Other disclosures pertaining to electronic or digital stethoscopes, such as disclosed in U.S. Pat. No. 5,774,563, teach the incorporation of digital and electronic functions for a stethoscope. Electronic stethoscopes offer features that allow the user to select whether to use the bell or the diaphragm of the stethoscope, switch between the conventional or electronic mode, to select a contact microphone, record a sound, play back a sound, alter the speed of a sound, or amplify or quiet the sound. However, due to the limited space on a stethoscope head it is difficult to incorporate features and controls into a stethoscope head without creating a stethoscope that is too cumbersome to operate.

Related to conventional acoustic stethoscopes with one or more sound frequency heads, such as a bell side and diaphragm side of a traditional two sided stethoscope head, a control barb is as a method of selecting between using the stethoscope bell or the stethoscope diaphragm on a dual mode stethoscope. The use of a stethoscope barb with a detent is a method used in the field to minimize the friction created between the stethoscope barb and the stethoscope head, which further facilitates the smooth rotation of the stethoscope barb. The prior teachings however fall short in disclosing a method for securing the detent to the control barb that does not require post connection alignment to ensure that the sound passageway remains unobstructed, or which allows for an unobstructed sound port through the detent and control barb.

This invention portrays an electronic stethoscope head that discloses a rotating digital control barb and corresponding electronics spool which are used in conjunction with a standard sized stethoscope head, and which digital control barb easily selects between multiple stopping points pursuant to the 360° rotation of the stethoscope head, and whereas the rotation sets a digital mode, and digital features, in addition to traditional acoustic bell and diaphragm selection through the incorporation of a second engagement track designated for the activation or deactivation of the digital features of an electronic stethoscope. This invention further teaches the method of securing a detent within the stethoscope head prior to insertion of the control barb into the stethoscope head such that post insertion alignment is not required to ensure unobstructed sound transmission, which method thereby saves in manufacturing time and cost.

### BRIEF SUMMARY OF THE INVENTION

In the preferred embodiment of the disclosed invention, a stethoscope head used in conjunction with a rotating digital control barb and electronics spool, whereto the elongated portion attaching to the stethoscope tubing is comprised of a rotating digital control barb and whereas the rotation of said digital control barb selects between mechanical and electrical positioning mode through a first engagement track. In the preferred embodiment of the disclosed invention, the rotating control barb utilizes separate stop points along a second track, through the 360° rotation of a stethoscope head, as a method of selecting conventional mode, digital mode, recording, playback, or amplification settings. Whereby in its preferred embodiment, the disclosed invention allows the user to select between digital features of the stethoscope, an incorporated contact microphone, or to use traditional features such as the acoustic bell or the diaphragm through the rotation of the digital control barb along said stop points which correlate to designated contacts within the electronics spool enclosed within the head of the stethoscope.

In the preferred embodiment of the disclosed invention, a digital control barb is disclosed in which in order to minimize friction, being caused from the surface of the digital control barb rubbing with the inner surface of the stethoscope head, a detent is secured to the end of the control barb being in contact with said inner surface, and whereas said detent is a cap being placed over said end of control barb prior to its insertion into the stethoscope head and whereby air ports are placed in said detent after said attachment to the control barb.

In another preferred embodiment of the present invention method of controlling the functions of a digital stethoscope through rotating a device along multiple stop points which is incorporated into the exterior rim of a stethoscope head, whereby the rim of the stethoscope head is rotated 360° along multiple stop points whereby a user selects between the features or controls of an electronic or digital stethoscope upon selecting certain stop points that correspond to specified digital contacts within the electronics spool.

### BRIEF DESCRIPTION OF THE SEVERAL VIEWS

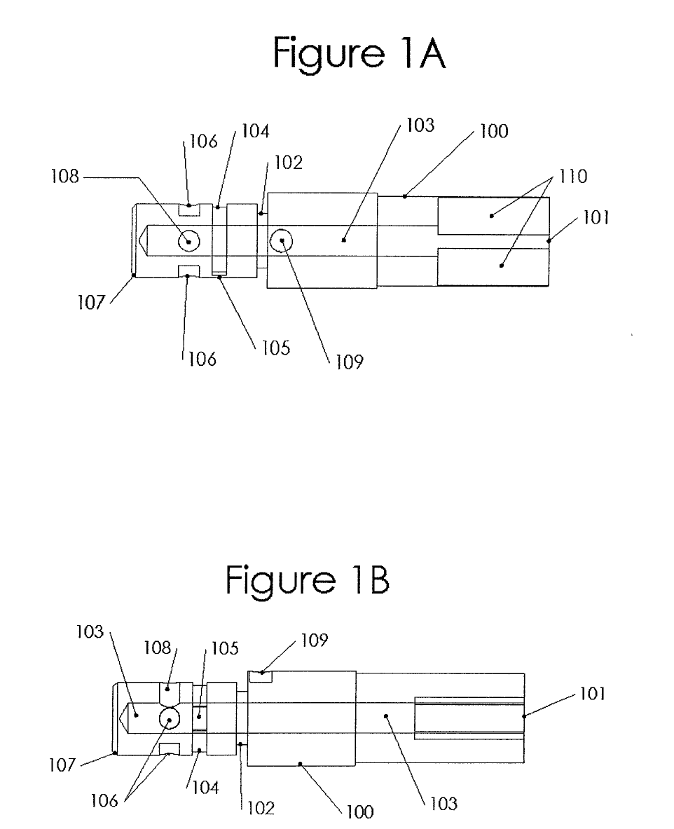

FIG. 1A shows a cross-sectional side view of the disclosed barb.

FIG. 1B shows a cross-sectional side view of the disclosed barb.

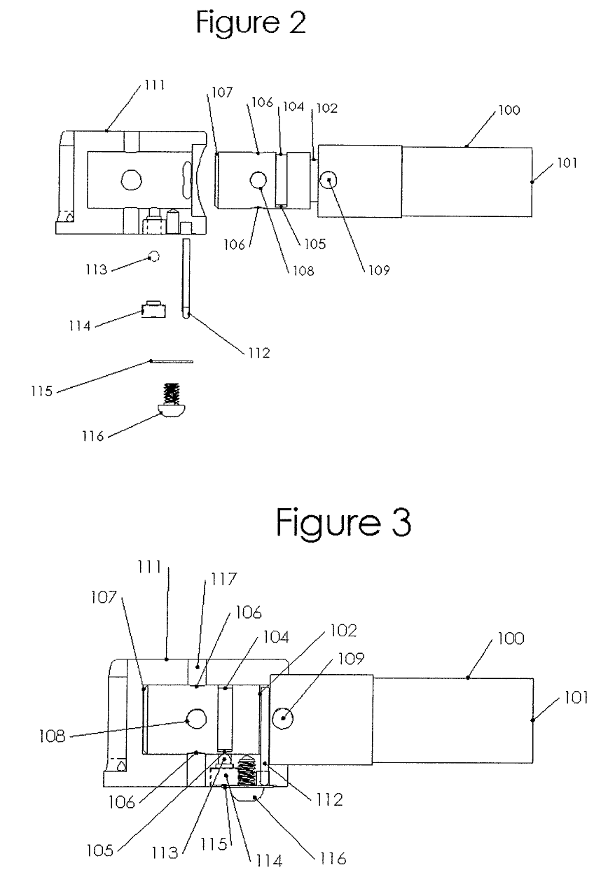

FIG. 2 shows an exploded cross-sectional side view the disclosed barb and interacting parts.

FIG. 3 shows a cross-sectional side view of the disclosed barb in connection with a corresponding barb spool.

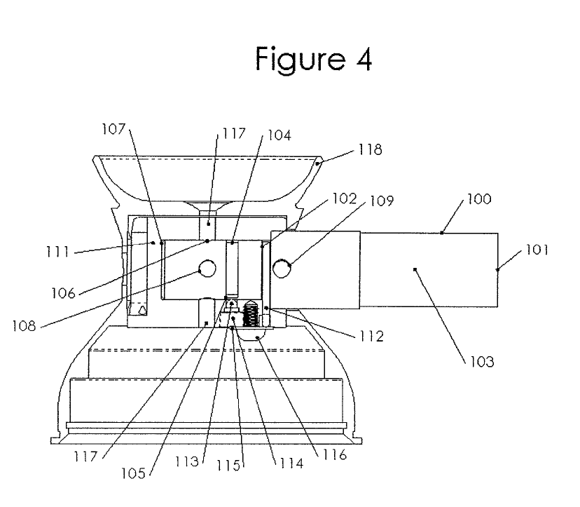

FIG. 4 shows a cross-sectional side view of the disclosed barb incorporated into a stethoscope shell.

### DETAILED DESCRIPTION OF THE INVENTION

Preferred embodiments of the present invention and their advantages may be understood by referring to FIGS. 1-4, like numerals being used for like corresponding parts in various drawings.

Referring to FIGS. 1A and 1B, a Barb 100 according to an embodiment of present invention is described. Barb 100 comprises an elongated shaft, which may be manufactured from medical grade stainless steel, titanium, or the like, and having Acoustic Channel 103 bored through the center of Barb 100 extending from open Proximal End 101, to be attached to stethoscope tubing using Receptacle 110, to the closed Distal End 107, which end is inserted into the head of a stethoscope, and whereby Barb 100 further comprises Locking Track 102 used as a means of securing Barb 100 to a stethoscope Spool 111, as shown in FIG. 2. Also shown in accordance with the preferred embodiment are two tracks comprised of an Engagement Track 104, a beveled cam following the circumference of Barb 100, and having Engagement Point 105, a raised portion along said Engagement Track 104. A second detent track being comprised of one or more Detent Holes 106 and having Acoustic Port 108, providing an path from the outside of Barb 100 through to Acoustic Channel 103. Further depicted are other embodiments of Barb 100 including Speaker Port 109 for providing an independent source of airflow to a speaker in an electronic stethoscope.

Referring to FIG. 2 an exploded view of the preferred embodiment of this invention, Barb 100 is shown along with and exploded view of Spool 111 and its connections for interacting with Barb 100. Specifically, Distal End 107 is shown as being inserted in Spool 111, and whereas Spool 111 is comprised of U-Clip 112 serving as a means of securing Barb 100 to Spool 111 by clamping to Barb 100 at Locking Track 102. FIG. 2 further shows the parts comprising the means of allowing the selectability of different electronic functions being offered and consisting of Activation Ball 113 that interacts with Engagement Point 105 located along Engagement Track 104 through being pressed into Activation Button 114 thereby enabling the desired function to be chosen, and further shown is Retaining Screw 116 and Washer 115 used in the preferred embodiment for holding U-Clip 112, Activation Button 114, and Activation Ball 113 to Spool 111.

Referring to FIG. 3, Barb 100 according to its preferred embodiment is shown in connection with Spool 111. Distal End 107 of Barb 100 sits within Spool 111 and is held within Spool 111 through U-Clip 112 being secured around Locking Track 102 located at near Distal End 107. The preferred embodiment of Barb 100 further depicts Engagement Track 104 and Engagement Point 105 being in line with Activation Ball 113, as well as Activation Button 114, Activation Ball 113, and U-Clip 112 being fastened to Spool 111 through Washer 116 and Retaining Screw 115. Barb 100 also comprises a second track consisting of multiple Detent Holes 106 and Acoustic Port 108, whereby said second track lines up with Acoustic Pathways 117, such that an unobstructed pathway is created from the outside of Spool 111 through Acoustic Port 108 when said Acoustic Port 108 is directly underneath Acoustic Pathway 117. Also shown is Air Port 109 located at the point where Spool 111 ends and which further comprises an air inlet that extends from outside Spool 111 to underneath Spool 111.

Referring to FIG. 4, the preferred embodiments of Barb 100 is shown as this invention interacts with Spool 111, and whereas Spool 111 is housed within a stethoscope Shell 118 containing certain electronic features. Barb 100 is permitted to spin along a 360° rotational axis, such that the rotation of Barb 100 within Spool 111 causes the simultaneous rotation of both Engagement Track 104 and the second track comprising Detent Holes 106 and Acoustic Port 108. Upon the rotation of Barb 100, Activation Ball 113 rolls along and within Engagement Track 104 until Activation Ball 113 contacts raised Engagement Point 105, and which contact thereby causes the raising Activation Ball 113 into Activation Button 114 thereby completing an integrated circuit and permitting the user to select a desired feature of the stethoscope. Additionally, the rotating of Barb 100 further causes the movement of Detent Holes 106 and Acoustic Port 108 of the second track, wherefrom Barb 100 is secured in one or more positions through the locking of a detent into corresponding Detent Hole 106, and whereby sound is permitted to travel from the desired Acoustic Pathway 117 through the Acoustic Port 108 upon Acoustic Port 108 being positioned directly under Acoustic Pathway 117.

What is claimed:

1. A rotating control barb comprising the elongated portion of a digital stethoscope head, whereas the rotating control barb provides for an acoustic path traveling lengthwise through the center of the control barb from the distal end to the proximal end, and having a means of attachment on the proximal end whereto the stethoscope tubing is securely fastened, and whereas the proximal end is connected to a stethoscope head having an electronic spool being incorporated into said stethoscope head, and whereby the rotating control barb along the 360° rotation of the elongated portion within the stethoscope allows a user to choose between the multiple features of a digital stethoscope through the incorporation of two or more engagement tracks, for the controlling of an electronic stethoscope's functions.

2. The rotating digital control barb whereby the rotating of said digital control barb along the 360° thereby rotates a first track having certain correlating engagement points within the electronics spool, such that said first engagement track moves underneath a engagement mechanism located within the inner surface of the electronics spool, and whereas the inner surface of said spool incorporates an electronic contact point that upon said engagement track activating said contact point thereby directing certain functions being incorporated into a digital stethoscope head to be utilized, and whereby said barb is closed on the distal end and having one or more acoustic ports and one or more detent holes that comprise a second engagement track on the lateral side of said distal end and further providing for an unobstructed path through said stethoscope barb for sound to be carried.

3. A rotating digital control barb of claim 2 that provides for a direct air channel to port a speaker enclosed within the stethoscope head not in connection with the diaphragm air port or bell air port.

4. The rotating control barb of claim 2 that selects whether the stethoscope is to operate in conventional mode, in which, the stethoscope operates using the acoustic bell or the acoustic diaphragm.

5. The rotating control barb of claim 2 which is able to set the digital mode of a stethoscope, which, thereby allows a user activate the electronics of digital stethoscope.

6. The rotating control barb of claim 2 that selects whether the stethoscope is operate by using the diaphragm or the acoustic bell of a digital stethoscope head, and that while the control barb is set to operate with acoustic functions, then the digital features are disengaged which prevents accidental destruction of recorded sound and excessive drain of the power supply.

7. The rotating control barb of claim 2 where the rotating control barb allows a user to select between multiple electronic functions offered by an electronic stethoscope, such as, but not limited to, the recording of sound, playing back sound, or sound amplification.

8. The rotating control barb of claim 2 where the rotating control barb incorporates mechanical switches along the two or more operations tracks.

9. The rotating control barb of claim 2, where the rotating control barb incorporates optical sensors along the two or more operations tracks.

10. The rotating control barb of claim 2, where the rotating control barb incorporates electromechanical or electromagnetic switches or sensors, such as but not limited to, halleffect, piezo, membrane, or proximity sensors.
